(19) United States
(12) Patent Application Publication
(10) Pub. No.: US 2023/0023230 A1
(43) Pub. Date: Jan. 26, 2023

(54) APPARATUS AND METHOD FOR REMOVING TISSUE FROM A BODY

(71) Applicant: Weinberg Medical Physics, Inc., North Bethesda, MD (US)

(72) Inventor: Irving N. WEINBERG, Chevy Chase, MD (US)

(21) Appl. No.: 17/815,071

(22) Filed: Jul. 26, 2022

Related U.S. Application Data
(60) Provisional application No. 63/225,839, filed on Jul. 26, 2021.

(51) Int. Cl. A61B 34/00; A61B 10/02; A61B 17/3205; A61B 90/96; A61B 17/32

(52) U.S. Cl. CPC A61B 34/73; A61B 10/0266; A61B 17/3205; A61B 90/96; A61B 17/320016; A61B 2034/2051

(57) ABSTRACT

Disclosed embodiments provide an apparatus and method of removing tissue from a body of a human or non-human animal. The disclosed embodiments includes a tool that may have a cutting edge to drill into tissue. The tool has magnetizable segments, which may be translated or rotated by an applied magnetic field

### CROSS REFERENCE AND PRIORITY CLAIM

This application claims priority to U.S. Provisional patent application Ser. No. 63/225,839, entitled “APPARATUS AND METHOD FOR REMOVING TISSUE FROM A BODY,” filed 26 Jul. 2021, the entirety of which is incorporated by reference.

### FIELD

Disclosed embodiments are directed to therapy or research involving humans or other animals, in particular apparatuses and methods for removing tissue from a body of humans or other animals.

### BACKGROUND

Removal of tissues from the body is often desired in order to inspect the tissues and thereby apply therapy to the body according to the nature of the tissues. Many types of biopsy tool and guns have been developed. Some biopsy tools use nested cylinders to capture a specimen of the tissue within a chamber. The specimen can then be removed from the interior of one of the cylinders manually.

Magnetic particles and tools have been used to deliver drugs. The magnetic tools may be pulled towards a magnet (as is usual for magnetizable materials) or may be pushed from a magnet, as described in U.S. Pat. No. 9,380,959 by Irving Weinberg, entitled “MRI-Guided Nanoparticle Cancer Therapy Apparatus and Technology”. Magnetic tools may be drilled into tissues, as described by L. Mair in U.S. Pat. No. 10,290,404, entitled “Method and Apparatus for Non-Contact Axial Particle Rotation and Decoupled Particle Propulsion”. Said drilling may be accomplished by applying a rotating magnetic gradient to a particle that has both axial and diametrically magnetized magnetic sections, so that the easy axis of the sections are different and their mechanical responses to magnetic fields are thereby different as well.

Disclosed embodiments provide a user with means of using magnetic fields to steer a tool (for example, a biopsy tool) into a location of interest in a body, to punch or drill the tool into tissue magnetically, to remove the tool (now containing tissue) from the body, and to remove the tissue from the tool for inspection. For the purposes of this specification, the terms “tool” and “biopsy tool” are used interchangeably, although it should be understood that the tool may have uses other than for biopsy (for example, to remove unwanted tissues).

### SUMMARY

Disclosed embodiments provide an apparatus and method of removing tissue from a body of a human or non-human animal. The disclosed embodiments includes a tool that may have a cutting edge to drill into tissue and a retaining edge to keep the tissue in the structure after biopsy has been performed. The tool has magnetizable segments which may be translated or rotated by an applied magnetic field, causing the tool to drill or push into tissue. The tool may travel to and from the tissue within a catheter, or without a catheter, from and to a location outside the body. Once outside the body, the tool may be opened by application of a magnetic field or through mechanical means, thereby releasing the tissue for inspection, disposal, or processing.

### BRIEF DESCRIPTION OF THE FIGURES

Aspects and features of the disclosed embodiments are described in connection with various figures, in which:

FIG. 1 illustrates biopsy tools;

FIG. 2 is an example demonstrating use of the biopsy tools according to the disclosed embodiments;

FIG. 3 is an example demonstrating how biopsy tools may be built according to the disclosed embodiments;

FIG. 4 is a flow chart of a method of performing a biopsy according to the disclosed embodiments;

FIG. 5 illustrates biopsy tools according to another embodiments; and

FIG. 6 illustrates alternative views of the biopsy tools according to the disclosed embodiments.

### DETAILED DESCRIPTION

The present invention will now be described in connection with one or more embodiments. It is intended for the embodiments to be representative of the invention and not limiting of the scope of the invention. The invention is intended to encompass equivalents and variations, as should be appreciated by those skilled in the art.

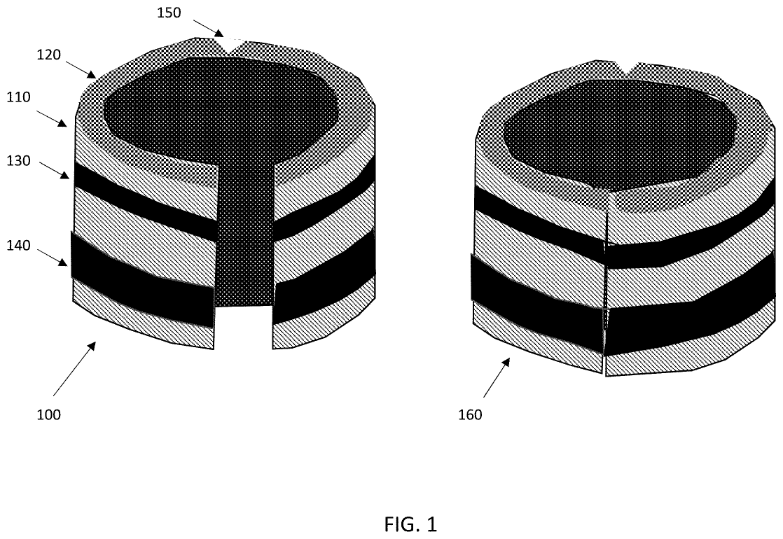

FIG. 1 shows two examples of embodiments of apparatus 100, which includes at least one biopsy tool 100. Tool 100 may be a band or other predominantly hollow structure, shown as an incomplete cylinder in FIG. 1. The tool has an inner diameter and an outer diameter. The outer diameter of the tool may be small enough (for example, 0.5 mm diameter or less) to fit in a mammary or prostate duct. The tool has a length, for example, of 3-15 mm. The tool includes one or more segments 130 and 140 of magnetizable material (for example, iron), and may include one or more segments of non-magnetizable material 110 (for example, plastic). A sharp edge 120 may be present to assist the tool in entering tissue. Edge 120 may be beveled or hooked so that the tissue is less likely to fall out of the tool during motion of the tool. A notch 150 or other feature that will result in a region of relative mechanical weakness may be present in the tool, which will subsequently assist in removing the tissue specimen.

Tool 160 illustrates an embodiment in which the cylinder is complete. The apparatus may include means of applying magnetic fields to the biopsy tool while the biopsy tool is in the body. The means of applying magnetic fields to the biopsy tool include current-carrying coils and/or permanent magnets, which may be operated under computer control. The means may be electropermanent magnet arrays that may be controlled to alternately image the biopsy tool and body and then propel the biopsy tool within the body, as for example taught in U.S. Pat. No. 10,908,240 B2 by Irving Weinberg, entitled “Method for Acquiring an Image and Manipulating Objects with Magnetic Gradients Produced by One or More Electropermanent Magnet Arrays” incorporated by reference in its entirety. See also, U.S. patent application Ser. No. 16/053,247 by Irving Weinberg and others, entitled “Apparatus, System and Methodologies for Biopsy or Removal of Tissues Using a Magnetically-Actuated Capsule,” the entirety of which is incorporated by reference.

It should be understood that the tool may have features that are recognizable optically or magnetically (i.e., bar coded), which may be useful in the case when many removals of tissues are desired by using multiple tools. The bar coding may be implemented through bands of magnetizable materials with different magnetic properties or physical dimensions.

It should be understood that the term “magnetizable material” means a material demonstrating ferromagnetic, ferrimagnetic, paramagnetic, or superparamagnetic ordering, and the term non-magnetizable material is a material not is not a magnetizable material.

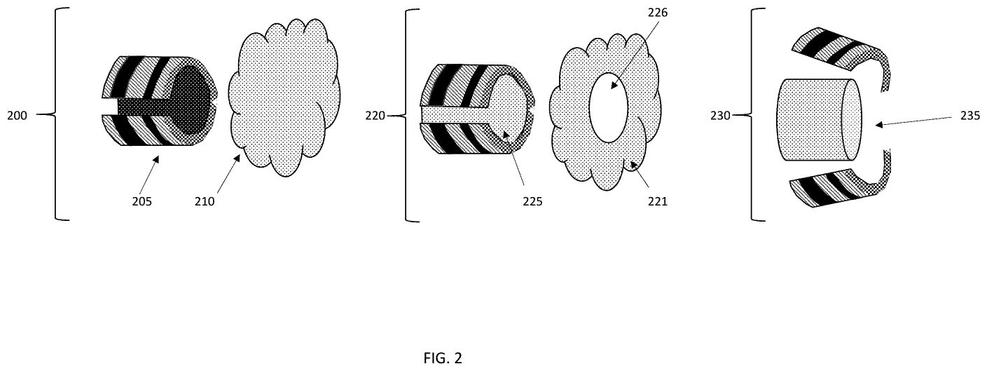

FIG. 2 shows an example of an embodiment of the method, starting with operation 200 in which the biopsy tool 205 of FIG. 1 is transported within less than 10 mm from a tissue 210 in a body. It should be understood that this transportation may be implemented by guiding the biopsy tool magnetically, for example through propulsion and/or rotation as described by Mair, U.S. Pat. No. 10,290,404, entitled “Method and apparatus for non-contact axial particle rotation and decoupled particle propulsion”, incorporated by reference. Alternatively, the transportation may be by means of a catheter containing the biopsy tool, said catheter tip approaching the tissue. The catheter may have a magnetic band at the tip, for example as in U.S. patent application Ser. No. 17/317,354 by Irving Weinberg, entitled “Magnetizable Clamp for a Catheter,” incorporated by reference. Immobilizing the catheter at one end may keep the catheter tip from moving when magnetic fields are applied to the biopsy tool. In an embodiment, both magnetic propulsion and/or rotation and a catheter may be used to bring the biopsy tool close to the tissue.

In operation 220, the biopsy tool 205 has drilled or plunged into the tissue 221, and a sample 225 of the tissue is now at least partially enclosed (i.e., captured) by the biopsy tool 205. Operation 220 shows that the removal of the biopsy sample 225 leaves a space 226 in the tissue 221. The space 226 left in the tissue 221 may or may not persist for seconds or longer periods of time after the sample 225 has been obtained by the biopsy tool, and mechanical relaxation of the tissue may cause the space 226 to close or significantly shrink over time. After operation 220 and prior to operation 230, the biopsy tool has been removed from the body, either by suction applied to a catheter surrounding at least part of the apparatus, or by propelling the biopsy tool, or by a combination of such methods or with some other method. In operation 230, a magnetic field or mechanical force or torque may be applied to the biopsy tool to release the sample 235. The notch or weak area 250 may assist in breaking the biopsy tool in a reliable manner. Operation 230 may be implemented in a machine that moves, examines (for example with optical or magnetic resonance microscopy) or otherwise processes the sample in an automated manner.

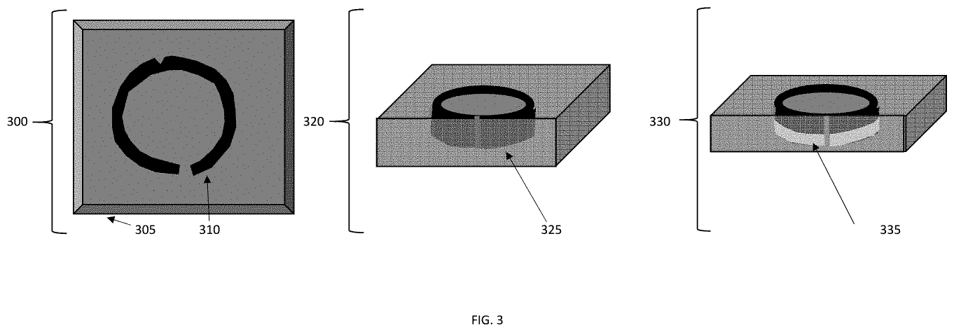

FIG. 3 illustrates a method for fabricating the apparatus. In operation 300, a material 305, for example a polyimide slab, may be etched with a laser or other cutting or etching tool to create a pattern 310. This state is also illustrated in the side view of 320, which demonstrates a band-shaped hole in the material 305. In operation 330, material 335 is deposited in the hole from the bottom up, for example with electroplating, as described in US Provisional Patent Application 63/219789 by Irving Weinberg, entitled “Apparatus and Method for Automated Manufacturing of Structures with Electrically Conductive Segments.”

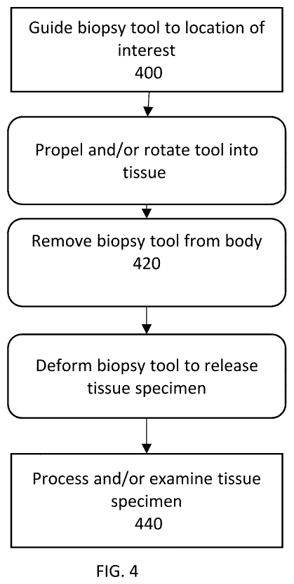

FIG. 4 describes the method of performing tissue removal beginning with guidance and/or propulsion of the biopsy tool to a location of interest near a tissue in a body 400. It should be understood that the biopsy tool may be the apparatus shown in FIG. 1. The biopsy tool is propelled and/or rotated into the tissue in 410. The biopsy tool is removed from the body 420, possibly by applying suction to a catheter that at some point contains the biopsy tool with enclosed biopsy sample, or possibly by applying force or tension to a wire connected to the biopsy tool or possibly with magnetic forces, or through a combination of these methods. Forces and/or torques are applied to the biopsy tool, releasing the tissue sample 430. In operation 440, the released tissue specimen is processed and/or examined.

It should be understood that some or all of the operations in FIG. 4 may be automated and under computer control.

It should be understood that the representation of the biopsy tool as a cylinder in the figures is not limiting, and the biopsy tool may be of other shapes that can at least partially enclose a portion of tissue.

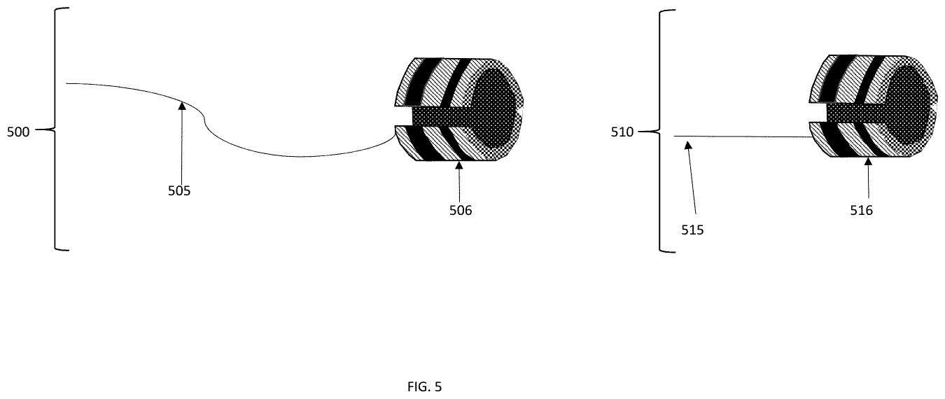

FIG. 5 shows two embodiments of the apparatus. In embodiment 500, the tool 506 has an attached flexible tether 505 that is flexible and may bend under strain or torque, for example if the tether is suture thread. The flexible tether 505 can be used to pull the tool 506 but is not effective at pushing the tool 506 or steering it in any way. In embodiment 510, the tool 516 has an attached rigid tether 515 which allows for no or minimal flexure when exposed to strain or torque. Unlike the flexible tether 505 in embodiment 500, the rigid tether 515 in embodiment 510 does allow for the application of pushing and torsional forces to the tool 516 and does allow a user to guide the tool 516 in both forwards, backwards, and steering directions, since the rigid tether 515 supports the application of both positive (pushing) and negative (pulling) forces on the tool 516.

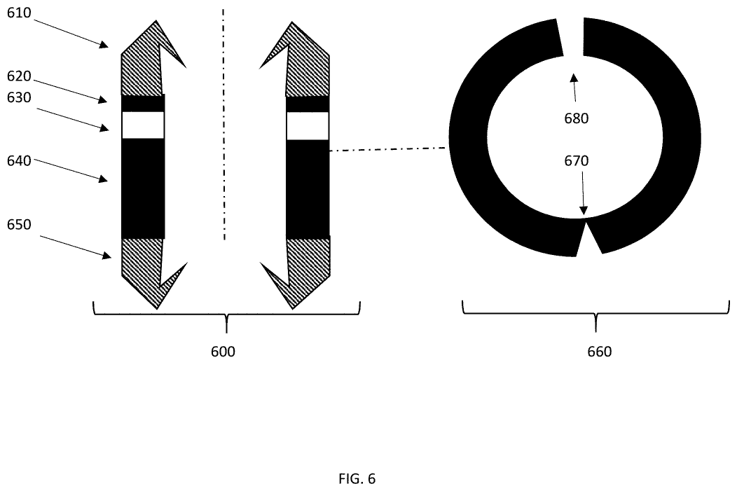

FIG. 6 is another representation of an embodiment of the apparatus in orthogonal views. The tool is shown in cross-sectional side view in 600, and in an orthogonal section in 660. In view 600, the cutting and retaining edge 610 is shown. A thin layer of magnetizable material 620 is shown. A non-magnetic layer 630 is shown. A thicker layer of magnetizable material 640 is shown. A second cutting and retaining edge 650 is shown. In the orthogonal section view 660, a notch 670 is present to weaken the wall of the tool so that the tool can be opened by applying a magnetic force or a mechanical force to the tool. A gap in the tool is shown as 680.

It should be understood that the apparatus may be used to remove tissue in situations other than biopsy, for example to debulk or remove a cancer or other unwanted tissue from a body. To better describe this general usage, the term “tool” is meant to include both biopsy tools (as described above) as well as the use of the apparatus for removing tissue from a body in situations other than biopsy. Similarly, the terms “tissue portion” and “sample” are used to include both biopsy specimens and other sections of tissue removed from a body.

Moreover, those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments but should instead be defined only in accordance with the following claims and their equivalents.

1. An apparatus comprising:
a tool containing at least one magnetizable material,
a magnet or coil or electropermanent magnet or arrays of such magnets, coils, or electropermanent magnets, to propel or rotate said tool within a body,
wherein the tool can be propelled or drilled into tissue by the magnet or coil or electropermanent magnet or arrays to capture a tissue and the tool can be deformed to release said tissue once outside the body.

2. The apparatus of claim 1, wherein the tool also contains at least one non-magnetizable material.

3. The apparatus of claim 1, wherein the tool is configured to capture a biopsy specimen.

4. The apparatus of claim 1, wherein the tissue is unwanted in the body.

5. The apparatus of claim 1, wherein the magnet or coil is part of an electropermanent magnet array that can image the tool and/or body.

6. The apparatus of claim 1, wherein the tool deformation is caused by a magnetic field.

7. The apparatus of claim 1, wherein the tool is magnetically or optically barcoded.

8. The apparatus of claim 1, wherein the tool has at least one edge to facilitate entry into tissue.

9. The apparatus of claim 1, wherein the tool has at least one edge to facilitate retention of the tissue in the tool until intended desired release of the tissue.

10. The apparatus of claim 1, wherein the tool has an attached tether, which may be flexible, semi-flexible, or rigid, configured to manipulate the tool.

11. The apparatus of claim 1, wherein the tool has an outer diameter of less than 0.5 mm.

12. The apparatus of claim 1, wherein the tool may be placed near the tissue portion by a catheter.

13. The apparatus of claim 12, wherein the catheter contains or is attached to a magnetizable material.

14. A method of removing tissue from a body, the method comprising:
propelling and/or drilling a tool containing magnetizable and non-magnetizable segments into tissue in the body using magnetic fields,
removing the tool from the body, and
deforming the tool to release the tissue captured by the tool.

15. The method of claim 14, wherein the magnetic fields are applied with at least one electropermanent magnet.

16. The method of claim 14, wherein the deformation is realized using magnetic fields.

17. The method of claim 14, wherein the deformation of the tool is realized by a force applied to open the tool via a notch formed in the tool.

18. The method of claim 14, wherein the tool has at least one edge to facilitate entry into tissue.

19. The method of claim 14, wherein the tool has at least one edge to facilitate retention of the tissue in the tool until intended desired release of the tissue.

20. The method of claim 14, wherein the tool has an attached tether, which may be flexible, semi-flexible, or rigid, configured to manipulate the tool.
